(19) United States
(12) Patent Application Publication
(10) Pub. No.: US 2005/0087197 A1
(43) Pub. Date: Apr. 28, 2005

(54) Method and apparatus for protecting a wounded area of a user's limb

(76) Inventor: Andrea Kelly, Lakewood, CO (US)

Correspondence Address: Dahl & Osterloth, LLP, Suite 3405, 555 17th Street, Denver, CO, 80202-3937, US

(21) Appl. No.: 10/692,760

(22) Filed: Oct. 24, 2003

(51) Int. Cl. A61F 5/37; A61F 13/06

(52) U.S. Cl. 128/892

(57) ABSTRACT

A kit for protecting a user's wounded limb from dirt or moisture comprises a tubular waterproof material, a fastener, and a strap. The fastener is fastened around a first end of the tubular waterproof material to form a waterproof sheath. The strap has portions that are adjustably coupled to one another to form a noose around a second end of the tubular waterproof material. The noose is tightened to secure the waterproof sheath to a user's limb. The components of the kit may alternately be provided to the user in partially assembled form, with some of the components being “sized to fit” prior to being given to the user.

### BACKGROUND

A person with a wounded limb often needs to protect their limb from exposure to dirt or moisture. Although various means exist for doing this, all have their drawbacks, and none have found wide acceptance.

### SUMMARY OF THE INVENTION

One aspect of the invention is embodied in apparatus comprising a tubular waterproof material, a fastener, and a strap. The fastener is fastened around a first end of the tubular waterproof material to form a waterproof sheath. The strap has portions that are adjustably coupled to one another to form a noose around a second end of the tubular waterproof material. The noose is tightened to secure the waterproof sheath to a user's limb.

Another aspect of the invention is embodied in a method comprising pulling a waterproof sheath over one's limb. The waterproof sheath comprises a tubular waterproof material that is secured at a first end via a fastener. A noose of a strap is slid over a second end of the waterproof sheath. The noose is formed by portions of the strap that are adjustably coupled to one another. One end of the strap is pulled to tighten the strap about the limb, and the noose frictionally engages the second end of the waterproof sheath as the end of the strap is pulled.

Other embodiments of the invention are also disclosed.

### BRIEF DESCRIPTION OF THE DRAWINGS

Illustrative embodiments of the invention are illustrated in the drawings, in which:

FIG. 1 illustrates a method for protecting a wounded area of a user's limb from exposure to dirt or moisture;

FIG. 2 illustrates a waterproof sheath pulled over a user's wounded limb;

FIG. 3 illustrates the noose of a strap slid over an end of the waterproof sheath shown in FIG. 2;

FIG. 4 illustrates the tightening of the strap shown in FIG. 3;

FIG. 5 illustrates a kit for supplying the items shown in FIGS. 3 & 4;

FIGS. 6-8 illustrate various embodiments of the strap shown in FIGS. 3-5;

FIG. 9 illustrates a method for supplying the items shown in FIGS. 3&4;

FIG. 10 illustrates the tubular waterproof material (referenced in FIG. 9) being dispensed from a roll; and

FIG. 11 illustrates a set of rolls of tubular waterproof material, from which the tubular waterproof material referenced in FIG. 9 may be selected.

### DETAILED DESCRIPTION OF THE INVENTION

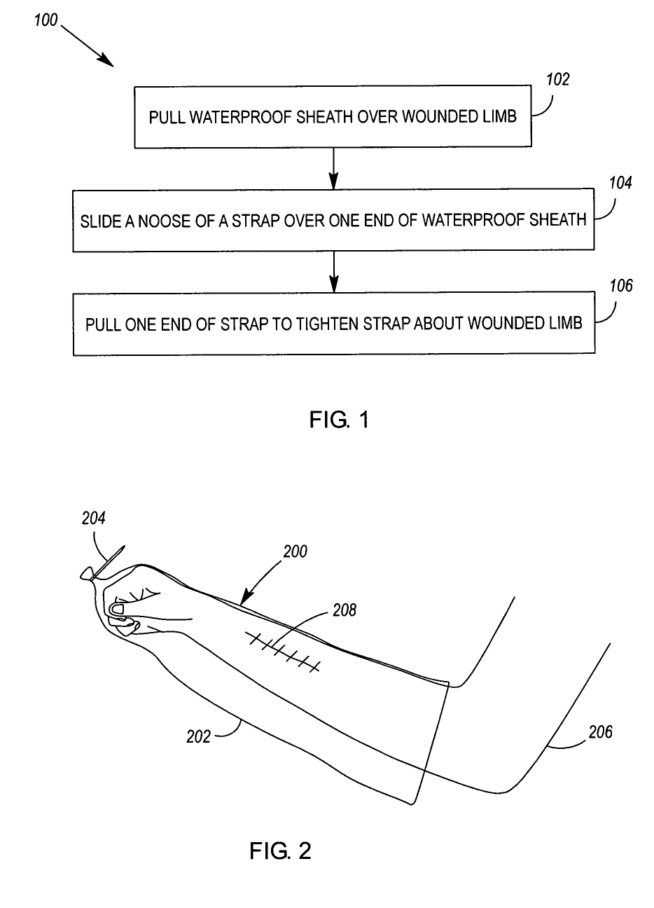

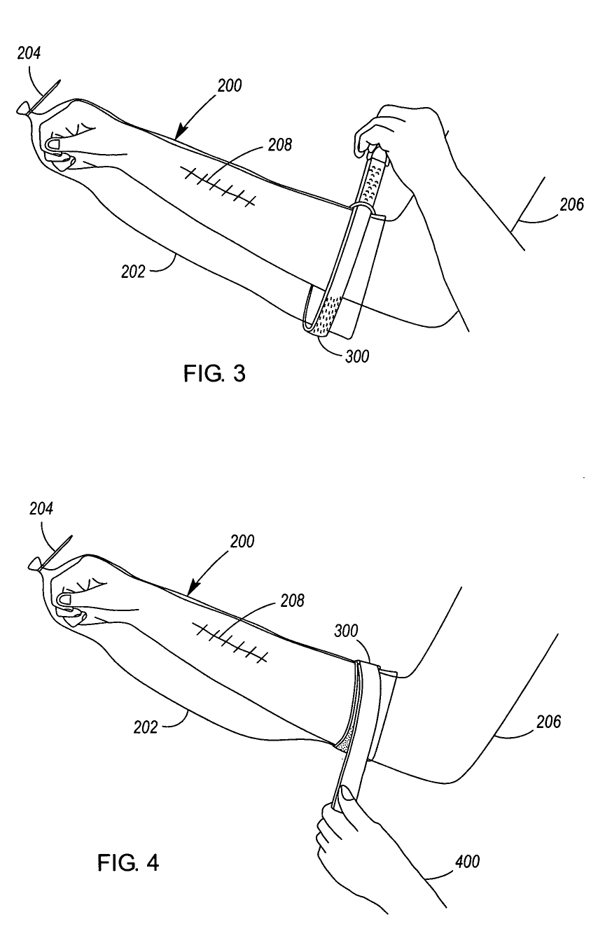

FIG. 1 illustrates an exemplary method 100 for protecting a wounded area of a user's limb from exposure to dirt or moisture. The method 100 commences with a user pulling 102 a waterproof sheath over a wounded limb. As shown in FIG. 2, the waterproof sheath 200 comprises a tubular waterproof material 202 that is secured at a first end via a fastener 204. After the user has pulled the waterproof sheath 200 over their limb 206, the user slides 104 a noose of a strap 300 over a second end of the waterproof sheath 200 (FIG. 3). As shown in FIGS. 6-8 (and as will be discussed later in this description), the noose is formed by portions of the strap that are adjustably coupled to one another. After positioning the strap 300, the user then pulls 106 one end of the strap 300 (using a free hand 400) to tighten the strap 300 about their limb 206, thereby sealing the wounded area 208 of their limb 206 within the waterproof sheath 200 (see FIG. 4). As the strap end is pulled, the noose frictionally engages the second end of the waterproof sheath 200. In this manner, the user may secure the waterproof sheath 200 to their limb 206 using only a single hand 400, and without the assistance of others. This convenience is especially important when the limb 206 to which the user is securing the waterproof sheath 200 is an arm and, thus, the user is left with but a single hand 400 to tighten the strap 300.

When a user is no longer in danger of exposing their wounded limb 206 to dirt or moisture, the strap 300 may be removed (e.g., by loosening the noose, or by decoupling the coupled portions of the strap 300), and the sheath 200 and strap 300 may be dried and stored for later reuse.

By way of example, the wounded area of a user's limb might comprise an area of an arm, hand, leg or foot having a cut, burn, cast, or site for administering intraveneous (IV) fluids.

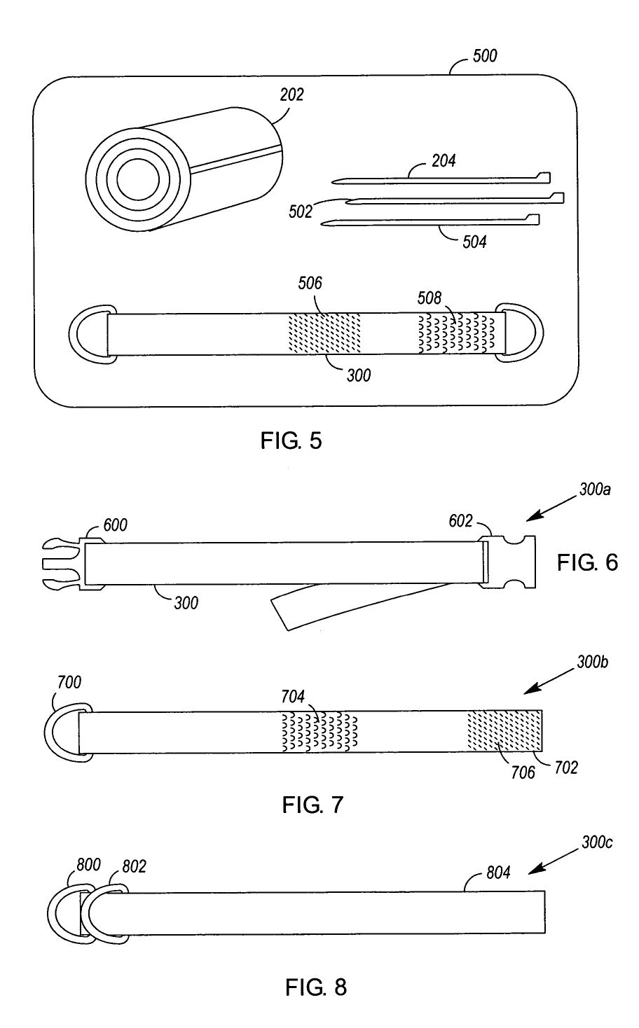

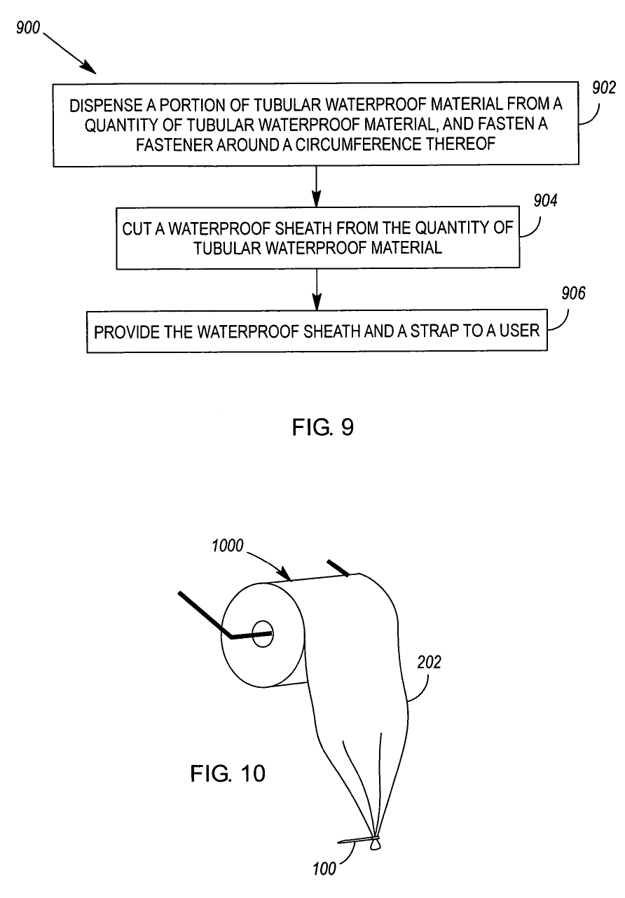

FIGS. 5 & 9 illustrate exemplary ways of supplying the waterproof sheath 200 (or components thereof) to a user. In FIG. 5, the waterproof sheath 200 is supplied in kit-form. By way of example, such a kit might be supplied by a grocery store, drug store, or medical supply store. In FIG. 9, the material of the waterproof sheath 200 is “sized to fit” (e.g., by a physician, nurse, pharmacist, or store clerk working at a hospital, emergency room, clinic or store) prior to being cut from a quantity (e.g., a roll or box) of tubular waterproof material.

Referring now to FIG. 5, one can see that exemplary contents of a kit 500 for protecting a wounded area of a user's limb from dirt or moisture may comprise a tubular waterproof material 202, a fastener 100, and a strap 300. In use, the tubular waterproof material 202 may be cut to a desired length such that it is long enough to cover a user's limb 206, up to and including a wounded area. However, depending on the length of tubular waterproof material 202 provided in the kit 500, a user may decide that the tubular waterproof material 202 may be used “as is” without cutting. The tubular waterproof material 202 may be formed of silicone, latex, Lycra, Anton or neoprene. By way of example, the strap 300 is shown to comprise a ring at each end. One ring is sized to fit (or is flexible enough to fit) through the other ring, as shown in FIGS. 2-4; and a hook-and-loop fastening system 506, 508 such as Velcro® is provided on the strap 300 so that it may be secured to itself when fastened about a user's limb.

The fastener 100 may take a variety of forms, such as a cable tie (shown), an elastic fastener (e.g., a rubberband or woven elastic band), or a clip. The fastener 100 may be fastened around a first end of a length of the tubular waterproof material 202, thereby forming a waterproof sheath 200 for insertion of a user's limb 206 (see FIGS. 2-4).

The strap 300 may also take a variety of forms, as shown in FIGS. 6-8. In each form 300a, 300b, 300c, the strap has portions that a user may couple to one another to form a noose. In FIG. 6, the potions that may be coupled are male and female buckle parts 600, 602. The strap 300a may then be tightened by pulling an end of the strap, and loosened by unfastening the buckle parts 600, 602.

In FIG. 7, the portions that may be coupled are 1) a ring 700, fixed to the strap 300b, and 2) an end 702 of the strap 300b that is sized to be pulled through the ring 700. A hook-and-loop fastening system 704, 706 such as Velcro® may then be used to secure the strap 300b.

In FIG. 8, the portions that may be coupled are 1) a pair of adjacent rings 800, 802, fixed to the strap 300c, and 2) an end 804 of the strap 300c that is sized to be woven through the rings 800, 802.

The rings 700, 800, 802 shown in FIGS. 7 & 8 may be of rigid (e.g., steel) or elastic (e.g., rubber) construction. If elastic, the ring(s) allow a strap to move as the muscles of a user's limb flex. By way of example, the strap 300 may be formed from a length of woven nylon. Alternately, the strap 300 could take other forms, and could be more or less elastic (e.g., the strap might be formed of rubber).

In use, the noose of the strap 300 frictionally engages a second end of the tubular waterproof material 202 1) after a user's limb 206 has been inserted into the waterproof sheath 200 and the noose has been placed around the second end, and 2) as one end of the strap 300 is pulled by the user to tighten the strap 300 about the user's limb 206 (FIG. 4). The waterproof sheath 200 is thereby sealed to the user's limb 206.

As previously indicated, if the strap 300b comprises but a single ring 700 through which an end 702 of the strap 300b is fed (FIG. 7), then the strap 300b may comprise a hook-and-loop fastening system 704, 706 (e.g., Velcro@) for securing the loose end 702 of the strap 300b to the circumference of the noose.

Although any of the straps 300a, 300b, 300c shown in FIGS. 6-8 may comprise a hook-and-loop fastening system 704, 706 for securing the loose end of the strap, the straps 300a, 300c shown in FIGS. 6 & 8 do not require such a system. This is because the securing systems of these straps maintain their tightened states after being secured by a user. The securing systems 600, 602, 800-804 shown in FIGS. 6 & 8 may therefore be advantageous in that a user may fasten them using a single, uni-directional pulling motion.

In one embodiment of the kit 500, the tubular waterproof material 202 is provided on a roll from which a plurality of lengths may be cut (thereby allowing the user to make a plurality of waterproof sheaths 200, as will be discussed below). In such a multi-use kit, a plurality of fasteners 100, 502, 504 may also be provided.

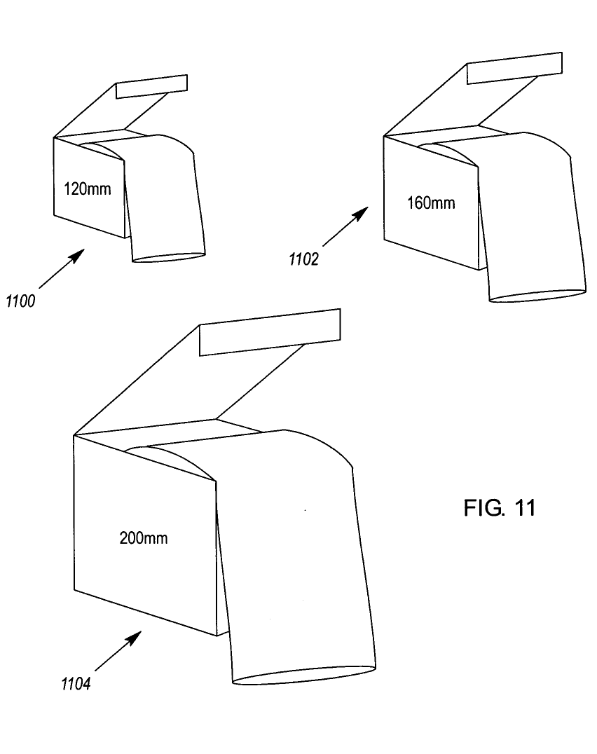

FIG. 9 illustrates a second exemplary way of supplying a waterproof sheath to a user. In the FIG. 9 method 900, the waterproof sheath is “sized to fit” prior to being cut from a quantity of tubular waterproof material. The method 900 comprises dispensing 902 a portion of tubular waterproof material from the quantity of said material, and then fastening a fastener around a circumference thereof. FIG. 10 shows the tubular waterproof material 202 being dispensed from a roll 1000, but the material could alternately be dispensed from a box or other container.

In one embodiment of the method 900, the tubular waterproof material is selected from a set of rolls 1100, 1102, 1104 of tubular waterproof material, each of which comprises a different diameter of tubular waterproof material (FIG. 11). In this manner, the diameter of tubular waterproof material may be selected based on the size of its intended user, as well as the size of any cast or other device which the waterproof material will need to cover when in use.

After attaching the fastener 100 to the tubular waterproof material 202, a waterproof sheath 200 is cut 904 from the quantity of material. The length of the waterproof sheath may be determined by comparing its length to what is needed to extend over a limb 206 and wounded area 208 of a particular patient (as shown in FIG. 2).

After forming the waterproof sheath 200, the sheath and a strap are provided to their user 906.

As previously discussed, one advantage of the invention is that a user with a wounded limb can fit a waterproof sheath to their limb, and then secure it with a strap, using only one hand. As a result, the assistance of others is not required. At the same time, the methods for providing the waterproof sheath allow the sheath to be easily cut to an appropriate length. Many past limb protectors have not been so easily “sized to fit”. Another advantage of the invention is that a low cost alternative is available for each of its components (i.e., the tubular waterproof material, fastener and strap).

While illustrative and presently preferred embodiments of the invention have been described in detail herein, it is to be understood that the inventive concepts may be otherwise variously embodied and employed, and that the appended claims are intended to be construed to include such variations, except as limited by the prior art.

1. A kit, comprising:
a) a tubular waterproof material that may be cut to a desired length;
b) a fastener which, when fastened around a first end of a length of the tubular waterproof material, forms a waterproof sheath for insertion of a user's limb; and
c) a strap for sealing the waterproof sheath to the limb of the user; the strap having portions that a user adjustably couples to one another to form a noose; the noose frictionally engaging a second end of the tubular waterproof material i) after the user's limb has been inserted into the waterproof sheath and the noose has been placed around the second end, and ii) as one end of the strap is pulled by the user to tighten the strap about the user's limb.

2. The kit of claim 1, wherein the fastener is a cable tie.

3. The kit of claim 1, wherein the fastener is an elastic fastener.

4. The kit of claim 1, wherein the strap comprises a hook-and-loop fastening system for securing the end of the strap pulled by the user to the circumference of the noose.

5. The kit of claim 1, wherein the portions of the strap that adjustably couple to one another comprise i) a ring, fixed to the strap, and ii) an end of the strap sized to be pulled through the ring.

6. The kit of claim 5, wherein the ring is elastic.

7. The kit of claim 5, wherein the strap comprises a hook-and-loop fastening system for securing the end of the strap pulled by the user to the circumference of the noose.

8. The kit of claim 1, wherein the portions of the strap that adjustably couple to one another comprise i) a pair of adjacent rings, fixed to the strap, and ii) an end of the strap sized to be woven through the rings.

9. The kit of claim 1, wherein the tubular waterproof material is provided on a roll from a plurality of said desired lengths may be cut; further comprising a plurality of said fasteners.

10. Apparatus, comprising:
a) a tubular waterproof material;
b) a fastener, fastened around a first end of the tubular waterproof material to form a waterproof sheath; and
c) a strap having portions that are adjustably coupled to one another to form a noose around a second end of the tubular waterproof material, thereby securing the waterproof sheath to a user's limb.

11. The apparatus of claim 10, wherein the fastener is a cable tie.

12. The apparatus of claim 10, wherein the fastener is an elastic fastener.

13. The apparatus of claim 10, wherein the strap comprises a hook-and-loop fastening system securing the end of the strap pulled by the user to the circumference of the noose.

14. The apparatus of claim 10, wherein the portions of the strap that adjustably couple to one another comprise i) a ring, fixed to the strap, and ii) an end of the strap sized to be pulled through the ring.

15. The apparatus of claim 14, wherein the ring is elastic.

16. The apparatus of claim 14, wherein the strap comprises a hook-and-loop fastening system securing the end of the strap pulled by the user to the circumference of the noose.

17. The apparatus of claim 10, wherein the portions of the strap that adjustably couple to one another comprise i) a pair of adjacent rings, fixed to the strap, and ii) an end of the strap sized to be woven through the rings.

18. A method, comprising:
a) dispensing a portion of tubular waterproof material from a quantity of tubular waterproof material, and fastening a fastener around a circumference thereof;
b) cutting a waterproof sheath from the quantity of tubular waterproof material; the waterproof sheath including the fastener; the length of the waterproof sheath being long enough to extend over a limb and wounded area of a particular patient; and
c) providing the waterproof sheath and a strap to the user; the strap having portions that the user adjustably couples to one another to form a noose; the noose frictionally engaging a second end of the tubular waterproof material i) after the user's limb has been inserted into the waterproof sheath and the noose has been placed around the second end, and ii) as one end of the strap is pulled by the user to tighten the strap about the user's limb.

19. The method of claim 18, further comprising, selecting the tubular waterproof material from a set of rolls of tubular waterproof material, each of the rolls comprising a different diameter of tubular waterproof material.

20. A method, comprising:
a) pulling a waterproof sheath over one's limb; the waterproof sheath comprising a tubular waterproof material that is secured at a first end via a fastener;
b) sliding a noose of a strap over a second end of the waterproof sheath; the noose being formed by portions of the strap that are adjustably coupled to one another; and
c) pulling one end of the strap to tighten the strap about the limb; the noose frictionally engaging the second end of the waterproof sheath as the end of the strap is pulled.
